(19) United States
(12) Patent Application Publication
(10) Pub. No.: US 2026/0256998 A1
(43) Pub. Date: Sep. 3, 2026

(54) OXYGENATOR

(71) Applicant: MAGASSIST CO., LTD., New District Suzhou, Jiangsu (CN)

(72) Inventors: Xianshan QI, Suzhou New District Suzhou, Jiangsu (CN); Dongxiao WANG, Suzhou New District Suzhou, Jiangsu (CN)

(73) Assignee: MAGASSIST CO., LTD., New District Suzhou, Jiangsu (CN)

(21) Appl. No.: 18/993,730

(22) PCT Filed: Apr. 21, 2023
(86) PCT No.: PCT/CN2023/089628

§ 371 Date: Jan. 13, 2025

(30) Foreign Application Priority Data
Jul. 13, 2022 (CN) 202210828527.8
Aug. 30, 2022 (CN) 202211053996.3

(51) Int. Cl. A61M 1/16

(52) U.S. Cl. CPC A61M 1/1698

(57) ABSTRACT

An oxygenator including a housing, first and second end covers, first and second sealing layers, an oxygenation module and a temperature control module is disclosed. The first and second end covers are respectively provided with first and second connecting ports, one of which being an oxygenation medium inlet, and the other being an oxygenation medium outlet. A first chamber and a second chamber respectively communicate with the first and second connecting ports. A side wall of the oxygenation module communicates with a blood inlet, and oxygenation fiber membranes of the oxygenation module have two ends respectively passing through the first and second sealing layers and respectively communicate with the first and second chambers. The temperature control module is located downstream of the oxygenation module in a blood flow direction, and has a side wall communicating with a blood outlet.

### FIELD

The present disclosure relates to an oxygenator.

### BACKGROUND

ECMO (Extracorporeal Membrane Oxygenation) is a medical device that performs gas exchange outside the body of a patient, serving as artificial heart and lungs to replace the functions of heart and lungs of the patient. It is commonly used in complex surgeries in the treatment of cardiac arrest, heart or lung failure, organ transplantation and the like.

As one of the core components of ECMO, oxygenators realize lung function by performing the exchange of carbon dioxide and oxygen in the blood. FIG. 1 shows an example of common membrane oxygenators. The blood of the patient is drawn out of the body and enters the oxygenator through a blood inlet, while fresh oxygen enters hollow oxygenation fiber bundles through a gas inlet. The fresh oxygen in the gas and the carbon dioxide in the blood on respective sides of the oxygenation fiber membranes exchange with each other through diffusion. Oxygenation efficiency (mL/min) is one of the important performance indicators of the oxygenator.

The oxygenator typically includes two layers of structures, namely heating fiber membranes and oxygenation fiber membranes. According to conventional knowledge in this technical field, an increase in temperature leads to higher oxygenation efficiency. Therefore, heating is usually performed before oxygenation to improve the oxygenation efficiency. Based on this knowledge, in the conventional structure described hereinabove, the heating fiber membranes are commonly arranged on the inside, while the oxygenation fiber membranes are arranged on the outside.

During the operation of the oxygenator, the blood passes through the spacing between the oxygenation fiber membranes, and a thickness of the oxygenation fiber membranes is equivalent to a flow length of the blood. Hence, the thickness of the oxygenation fiber membranes is vital to the oxygenation efficiency. Generally, there is a positive correlation between the oxygenation efficiency and the thickness of the oxygenation fiber membranes. A new technical problem in this technical field is how to achieve higher oxygenation efficiency under same fabricating costs, or in other words, how to maximize the oxygenation efficiency with a same volume of the oxygenation fiber membranes.

Additionally, blood pressure drop (mmHg) is another parameter as important as the oxygenation efficiency. The blood being drawn out of the patient body undergoes oxygenation in the oxygenator before returning to the patient body. During this process, the pressure of the flowing blood decreases due to energy loss or flow resistance, which is referred to as the blood pressure drop. In this technical field, it is expected that the oxygenator will cause as low a blood pressure drop as possible on the premise of ensuring relatively high oxygenation efficiency.

Moreover, the extracorporeal circulation is expected to involve a relatively small volume of blood during the operation of the oxygenator. For example, if an oxygenator requiring a large blood perfusion volume is applied to a patient with anaemia, or a patient of small body size, such as a child, extreme cases may occur where all the blood of the patient is involved in the extracorporeal circulation, or the perfusion requirement of the oxygenator cannot be satisfied even if all the blood of the patient is involved in the extracorporeal circulation. Apparently, such cases are undesirable. Additionally, if the blood perfusion volume of the oxygenator is relatively large, then a large amount of priming fluid is also required during the priming stage before the surgery. This greatly prolongs the priming time, affecting the preparation for the surgery. Therefore, it is a clinical requirement to improve the structure of the oxygenator to minimize the blood perfusion volume.

Furthermore, the blood flows in two roughly perpendicular directions, i.e., the blood flows into the oxygenation fiber membranes while flowing forward to the downstream area, entering the oxygenation fiber membranes downstream. When flowing downstream, the blood inevitably experiences pressure loss, which further causes uneven pressure at different positions where the blood enters the oxygenation fiber membranes.

### SUMMARY

In view of this, an oxygenator is provided according to the present disclosure, which mitigates at least one of the problems described hereinabove.

To solve the above technical problems, the oxygenator according to the present disclosure includes a housing, a first end cover and a second end cover that are respectively arranged at both ends of the housing, a first sealing layer and a second sealing layer that are formed in the housing, and an oxygenation module and a temperature control module arranged in the housing. The first end cover and the second end cover are respectively provided with a first connecting port and a second connecting port. One of the first connecting port and the second connecting port is an oxygenation medium inlet, and the other one of the first connecting port and the second connecting port is an oxygenation medium outlet. A first chamber is defined by the first sealing layer and the first end cover, and is in communication with the first connecting port. A second chamber is defined by the second sealing layer and the second end cover, and is in communication with the second connecting port. A side wall of the oxygenation module is in communication with a blood inlet, and the oxygenation module includes an oxygenation fiber membrane. Two ends of the oxygenation fiber membrane pass through the first sealing layer and the second sealing layer to be in communication with the first chamber and the second chamber respectively. The temperature control module is located downstream of the oxygenation module in a flow direction of blood, and a side wall of the temperature control module is in communication with a blood outlet.

Preferably, the first end cover is provided with a third connecting port, and the second end cover is provided with a fourth connecting port. One of the third connecting port and the fourth connecting port is a temperature control medium inlet, and the other one of the third connecting port and the fourth connecting port is a temperature control medium outlet. A third chamber is defined by the first sealing layer and the first end cover, and is fluidly isolated from the first chamber. A fourth chamber is defined by the second sealing layer and the second end cover, and is fluidly isolated from the second chamber. The temperature control module includes a temperature control fiber membrane. Two ends of the temperature control fiber membrane pass through the first sealing layer and the second sealing layer to be in communication with the third chamber and the fourth chamber respectively.

Preferably, the blood inlet is provided on the first end cover. The oxygenation module is substantially of a cylindrical structure, and an inner side wall of the oxygenation module is in communication with the blood inlet. The temperature control module is substantially of a cylindrical structure, and is arranged on an outer side of the oxygenation module. An outer side wall of the temperature control module is spaced apart from an inner side wall of the housing to form a gap space therebetween, and the gap space is in communication with the blood outlet.

Preferably, the blood outlet is provided on a side wall of the housing, and an axis of the blood outlet is located on an inner side of a tangent line. The tangent line and the axis are located on the same side of a central axis of the housing. The tangent line is parallel to the axis and is tangent to an outer wall of the housing. An offset distance between the tangent line and the axis preferably ranges from 2 cm to 10 cm.

Preferably, an acute corner section is formed between the blood outlet and the housing, and a round corner or an arc transition is provided at the acute corner section.

Preferably, a substantially dome-shaped structure that protrudes outwards is formed on the first end cover is formed on the first end cover. The blood inlet is in communication with the dome-shaped structure, and the dome-shaped structure is provided with a gas vent.

Preferably, no any structure is provided between the oxygenation module and the temperature control module.

Preferably, a ratio L/H of a thickness L of the oxygenation module in a radial direction to a height H of the oxygenation module in an axial direction is between 0.525 and 1.562.

Preferably, a first isolator is provided in the housing, and the oxygenation fiber membrane is wound around the first isolator. The first isolator is of a hollow cylindrical structure. An internal space of the first isolator is in communication with the blood inlet, and a side wall of the first isolator is provided with a first hole for the blood to pass through. A second isolator is provided in the housing and is located between the oxygenation module and the temperature control module. The temperature control fiber membrane is wound around the second isolator, and a side wall of the second isolator is provided with a second hole for the blood to pass through. A ratio of a volume of the first hole to a volume of a space occupied by the first isolator is α1, and a ratio of a volume of the second hole to a volume of a space occupied by the second isolator is α2, and α1>α2. Specifically, a value of α1 is between 0.452 and 0.951, and a value of α2 is between 0.311 and 0.849.

Preferably, a diversion cone is provided in the housing and passes through the first isolator. In a direction from the second end cover to the first end cover, a gap distance between an outer wall of the diversion cone and an inner wall of the first isolator gradually decreases.

Preferably, a circumferential flange is formed on the first end cover and extends to the first sealing layer. One end of the first isolator is connected to the diversion cone, and the other end of the first isolator is connected to the circumferential flange. A blood guiding chamber is defined by the circumferential flange and the first isolator, and accommodates the diversion cone. The blood guiding chamber includes a blood inlet region, and a part of the diversion cone extends into the blood inlet region, where the blood inlet region is a region in the blood guiding chamber between the first end cover and a surface cross section of the first sealing layer away from the first end cover.

Preferably, a ratio of a volume of the part of the diversion cone extending into the blood inlet region to a volume of the blood inlet region is between 0.293 and 0.726. The diversion cone includes a first cone section that is close to the first end cover, and at least a part of the first cone section is located in the blood inlet region. A cone tip of the first cone section passes through the first sealing layer into the circumferential flange, and a distance between the cone tip and a top of the blood inlet region is between 0.012 cm and 0.546 cm. A ratio of the distance between the cone tip and the top of the blood inlet region to a height of the first cone section is between 0.009 and 0.237.

Preferably, the diversion cone further includes a second cone section that is close to the second end cover and is connected to the first cone section, and a part of the second cone section is located in the first isolator. A cone angle of the first cone section is greater than a cone angle of the second cone section.

Preferably, a minimum effective flow area of the blood inlet region is larger than or equal to a cross-sectional area of the blood inlet.

According to the conventional knowledge, the oxygenation efficiency is improved when the temperature of the blood is increased. Thus, in the conventional structure, the blood flowing through the oxygenator is heated before oxygenation. Generally, the longer the flow length of the blood in the oxygenation fiber membranes, the higher the oxygenation efficiency. Hence, improvements to the conventional technology are limited to the idea of extending the flowing length of the blood in the oxygenation fiber membranes by increasing the thickness of the oxygenation fiber membranes, so as to achieve higher oxygenation efficiency. However, this leads to higher costs. According to the present disclosure, the oxygenation module is arranged upstream without increasing the volume of the oxygenation fiber membranes. By reducing the inner diameter of the oxygenation module, the flow length of the blood in the oxygenation module becomes longer, so that the oxygenation efficiency is enhanced. That is, higher oxygenation efficiency is achieved with the same volume of oxygenation fiber membranes (i.e. same costs for the oxygenation fiber membranes). Or, less volume of oxygenation fiber membranes is needed (i.e. the costs of the oxygenation fiber membranes are reduced) for the same oxygenation efficiency.

Pressure drop is inevitable when the blood flows in the oxygenator. The blood pressure drop is as important an indicator as the oxygenation efficiency. The blood pressure drop increases with the flow length of the blood in the oxygenation module. The technical solutions according to the present disclosure seek a balance between the oxygenation efficiency and the pressure drop on the basis of the first improvement. When the volume and height of the oxygenation fiber membranes keep constant, the ratio of the width to the height of the oxygenation fiber membranes is adjusted to decrease the blood pressure drop while maintaining the oxygenation efficiency.

### BRIEF DESCRIPTION OF THE DRAWINGS

FIG. 1 is a schematic structural view of a hollow fiber membrane oxygenator according to the conventional technology;

FIG. 2 is a schematic structural view of an internal flow passage of the oxygenator according to the conventional technology;

FIG. 3 is a curve showing the relationship between oxygenation efficiency and pressure drop;

FIG. 4 is a perspective view of an oxygenator according to a preferred embodiment of the present disclosure;

FIG. 5 is a top view of the oxygenator shown in FIG. 4;

FIG. 6 is a side view of the oxygenator shown in FIG. 4;

FIG. 7 is a sectional view taken along a line A-A in FIG. 6;

FIG. 8 is a sectional view taken along a line C-C in FIG. 5;

FIG. 9 is a schematic view showing a flowing state of an oxygenation medium;

FIG. 10 is a sectional view taken along a line B-B in FIG. 6;

FIG. 11 is a sectional view taken along a line D-D in FIG. 6;

FIG. 12 is a schematic structural view of a diversion cone;

FIG. 13 is a sectional view of another embodiment of the present disclosure showing a blood outlet; and

FIG. 14 is a sectional view of an oxygenator according to further another embodiment of the present disclosure.

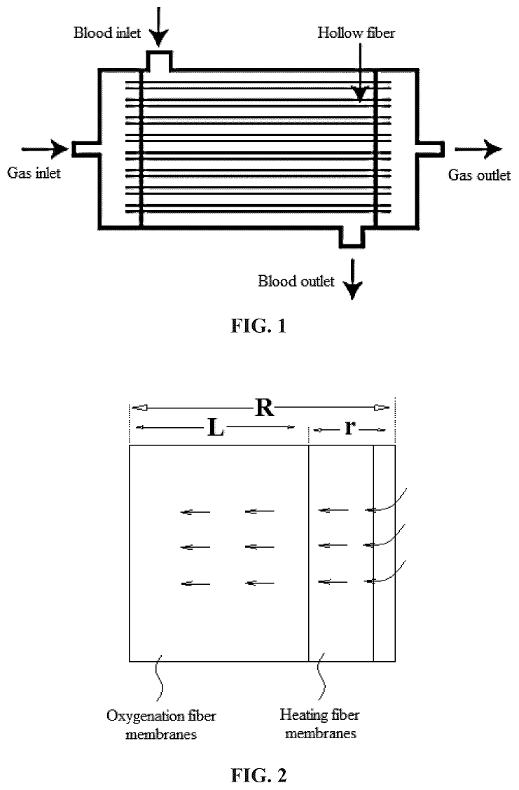

### DETAILED DESCRIPTION OF THE EMBODIMENTS

The embodiments of the present disclosure will be described hereinafter with reference to the drawings. Those skilled in the art may understand that, various modifications may be made to the embodiments being described herein without departing from the spirit and scope of the present invention. Therefore, the drawings and description are essentially illustrative, and are not intended to limit the protection scope of the claims. Additionally, in this specification, the drawings are not drawn to scale, and the same reference numerals represent the same parts.

It should be noted that, the terms “first” and “second” in the description of the embodiments of the present disclosure are for distinguishing between two different entities or parameters with the same name. Thus, the terms “first” and “second” are merely for the convenience of description, and should not be construed as limitations to the embodiments of the present disclosure, which will not be described in detail in the subsequent embodiments.

Based on the conventional knowledge of the impact of temperature on oxygenation efficiency, as shown in FIG. 2, in a typical conventional embodiment, fiber membranes of an oxygenator form a double-layer cylindrical structure. A heating fiber membrane layer is provided on the inside, and an oxygenation fiber membrane layer is provided on the outside. Trajectory lines with arrows in the drawing illustrate a flow direction of blood. The blood flows to the heating fiber membranes, and then flows to the oxygenation fiber membranes, thereby finishing the oxygenation process.

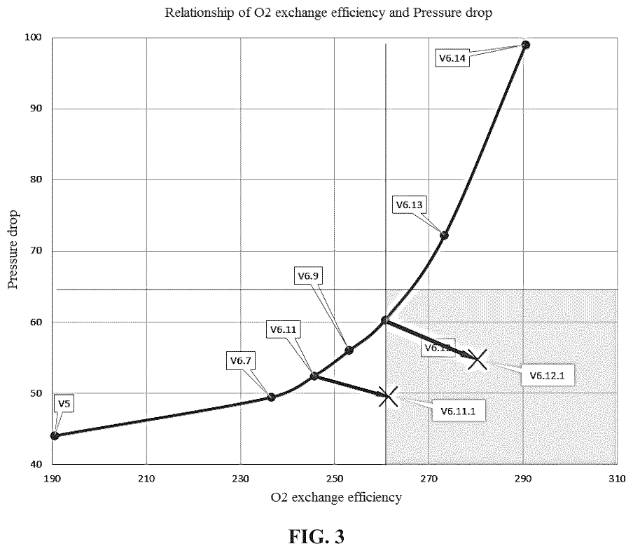

Studies have shown that, there is a relationship between oxygenation efficiency and blood pressure drop of the oxygenator, which is illustrated by a curve in FIG. 3. It can be observed that, the blood pressure drop increases with the oxygenation efficiency. For example, when the oxygenation efficiency is above 270 mL/min, the increase of the blood pressure drop is significantly enhanced. According to the results shown in FIG. 3, it is expected in a preferred embodiment of the present disclosure that, the oxygenator is designed to achieve the highest oxygenation efficiency within a reasonable pressure drop range. That is, the advantage of high oxygenation efficiency and low blood pressure drop is achieved in a shaded area in FIG. 3.

As shown in FIG. 2, in the double-layer cylindrical structural design of the oxygenator, an outer diameter of the oxygenation fiber membrane layer is R, and an inner diameter of the oxygenation fiber membrane layer is r. A flow length of the blood in the oxygenation fiber membrane layer, i.e., a thickness L of the oxygenation fiber membrane layer in a radial direction satisfies the following relationship:

$$L = R - r \qquad Equation ( 1 )$$

A volume V of the oxygenation fiber membranes and a height H of the oxygenation fiber membrane layer in an axial direction satisfy the following relationship:

$$H = V / [ π ( {R}^{2} - {r}^{2} ) ] \qquad Equation ( 2 )$$

Based on the Equations (1) and (2), in consideration of a positive correlation between the oxygenation efficiency and the thickness of the oxygenation fiber membranes, in theory, higher oxygenation efficiency can be realized by increasing the thickness L of the oxygenation fiber membrane layer. Based on Equation (1), when the volume V and the height H of the oxygenation fiber membranes remain constant, the thickness L of the oxygenation fiber membrane layer can be increased by reducing the inner diameter r of the oxygenation fiber membrane layer.

However, according to the conclusion from FIG. 3, the blood pressure drop increases with the oxygenation efficiency. Therefore, in order to balance the oxygenation efficiency and the blood pressure drop, it is not simply about increasing the thickness L of the oxygenation fiber membrane layer. Instead, the above object is achieved by adjusting a ratio L/H.

In view of this, when the volume V and the height H of the oxygenation fiber membranes remain constant, the inner diameter r of the oxygenation layer can be reduced by arranging the oxygenation module on the inside. Guided by this technical essence, the temperature control module is arranged downstream of the oxygenation module in the flow direction of the blood. In this way, contrary to the conventional knowledge, the blood entering the oxygenator undergoes oxygenation before temperature control.

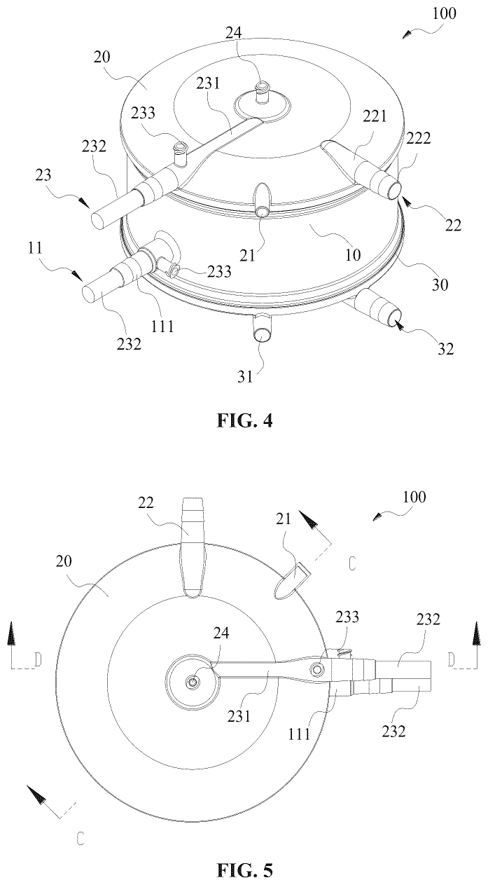

As shown in FIGS. 4 and 5, an oxygenator 100 according to the present embodiment includes a hollow housing 10 with openings at both ends thereof, and a first end cover 20 and a second end cover 30 that respectively cover the openings at the both ends of the housing 10. The first end cover 20 and the second end cover 30 are respectively assembled at a first end and a second end of the housing 10. After the covering and fixations, an outer profile structure of the oxygenator is formed.

Two main working modules are provided in the hollow housing 10, namely an oxygenation module, which is for an oxygenation medium to flow through to oxygenate venous blood or oxygen-depleted blood, and a temperature control module for regulating a temperature of the blood. The oxygenation module includes multiple wound oxygenation fiber membranes. Internal passages of the oxygenation fiber membranes are for the oxygenation medium, such as oxygen, to pass through, and the blood is oxygenated when flowing through a spacing between the oxygenation fiber membranes. The temperature control module regulates the temperature of the blood by increasing, decreasing and preserving the temperature, and may employ any proper conventional structures, such as electric heating or a water bath coil. Or, the temperature control module may have a structure similar to the oxygenation module. That is, the temperature control module is formed by multiple wound temperature control fiber membranes, and the temperature control fiber membranes are filled with a temperature control medium, such as water, thereby regulating the temperature of the blood flowing through. A temperature of the water may be regulated according to the temperature control needs. For example, hot water is used for increasing the temperature.

An internal flow passage of the oxygenator 100 are divided into three parts that are isolated from each other, which are:
1) the oxygenation medium enters through an inlet, passes through the internal passages of the oxygenation fiber membranes, and departs through an outlet;
2) the temperature control medium enters through another inlet, passes through internal passages of the temperature control fiber membranes, and departs through another outlet; and
3) the blood enters through further another inlet, passes through the oxygenation module and the temperature control module in the listed sequence, and flows out of further another outlet.

The internal passage of each oxygenation fiber membrane forms a part of an oxygenation gas passage, and the internal passage of each temperature control fiber membrane forms a part of a temperature control flow passage. The blood passes through the spacing between the oxygenation fiber membranes, i.e., passes between outer side walls of parts of the oxygenation gas passage to be oxygenated. Similarly, the blood passes through another spacing between the temperature control fiber membranes, i.e., passes between outer side walls of parts of the temperature control flow passage, so that the temperature of the blood is regulated. As described hereinabove, the temperature control module is downstream of the oxygenation module in the flow direction of the blood. Different from the conventional technology and knowledge, the blood undergoes the oxygenation before the temperature control when flowing through the oxygenator according to the present embodiment.

As shown in FIG. 4, a first connecting port 21 and a second connecting port 31 are respectively formed on the first end cover 20 and the second end cover 30, and are both in communication with the oxygenation gas passage. One of the first connecting port 21 and the second connecting port 31 is an oxygenation medium inlet, and the other one of the first connecting port 21 and the second connecting port 31 is an oxygenation medium outlet. Based on the orientation shown in FIG. 4, the first end cover 20 is arranged at a top of the housing 10, and the second end cover 30 is arranged at a bottom of the housing 10. Schematically, the first connecting port 21 is the oxygenation medium inlet, and the second connecting port 31 is the oxygenation medium outlet.

In an embodiment where the temperature control of the blood is realized by filling the temperature control medium into the temperature control fiber membranes, a third connecting port 22 and a fourth connecting port 32 are further formed on the first end cover 20 and the second end cover 30 respectively. The third connecting port 22 and the fourth connecting port 32 are both in communication with the temperature control flow passage. One of the third connecting port 22 and the fourth connecting port 32 is a temperature control medium inlet, and the other one of the third connecting port 22 and the fourth connecting port 32 is a temperature control medium outlet. The third connecting port 22 includes an extension portion 221 that is located on the first end cover 20 and a temperature control connector end 222 that extends outwards from the extension portion 221. The extension portion 221 is recessed in a surface of the first end cover 20 at an edge of the first end cover 20, and the temperature control connector end 222 is configured to connect to a temperature control pipeline. The structure of the fourth connecting port 32 is basically the same as the structure of the third connecting port 22, and is not described in detail herein. Based on the orientation shown in FIG. 4, the fourth connecting port 32 serves as the medium inlet and is located at the bottom, and the third connecting port 22 serves as the medium outlet and is located at the top. That is, the temperature control medium flows from bottom to top in a direction opposite to a flow direction of the oxygenation medium.

A blood inlet 23 is formed on the first end cover 20. The blood inlet 23 includes an extension portion 231 and a connector end 232 extending outwards from the extension portion 231. The extension portion 231 extends in a radial direction from a center of the first end cover 20, and is recessed in the surface of the first end cover 20. The connector end 232 is configured to connect to blood tubing. A position of a blood outlet 11 may be determined according to actual needs. For example, the blood outlet 11 may be arranged on the second end cover 30 or on a side wall of the housing 10. The blood outlet 11 includes an extension portion 111 and a connector end 232. The extension portion 231 of the blood inlet 23 and the extension portion 111 of the blood outlet 11 are each provided with an infusion connecting port 233 for an infusion of an anticoagulant into the blood entering and departing from the oxygenator 100.

As shown in FIG. 5, a gas vent 24 is formed on the first end cover 20, and a waterproof breathable membrane (not shown in the drawing) is provided in the gas vent 24. As shown in FIG. 7, the extension portion 231 of the blood inlet 23 is eccentric relative to a blood inlet region of a blood flow passage in the oxygenator 100. After entering the oxygenator 100, the blood swirls in the blood flow passage. Under the action of a centrifugal force, bubbles are separated from the blood, pass through the waterproof breathable membrane, and are discharged through the gas vent 24.

An internal structure of the oxygenator 100 is divided into three layers from the center to the periphery, namely a diversion cone 40 at an inner layer, the temperature control module 60 at an outer layer, and the oxygenation module 50 between the diversion cone 40 and the temperature control module 60. This triple-layer structure is partitioned by isolators. Specifically, the diversion cone 40 is isolated from the oxygenation module 50 by a first isolator 70 that is provided with a first hole. The oxygenation fiber membranes of the oxygenation module 50 is wound around the first isolator 70. The first isolator 70 is substantially cylindrical, and the diversion cone 40 passes through the first isolator 70. A gap is formed between the diversion cone 40 and the first isolator 70, and the gap is in communication with a side wall of the oxygenation module 50 through the first hole. The oxygenation module 50 is isolated from the temperature control module 60 by a second isolator 80 that is provided with a second hole. The temperature control fiber membranes of the temperature control module 60 are wound around the second isolator 80, and the oxygenation module 50 is in communication with the temperature control module 60 through the second hole. Another gap between the temperature control module 60 and the housing 10 forms a gap space, and the gap space is in communication with the blood outlet 11.

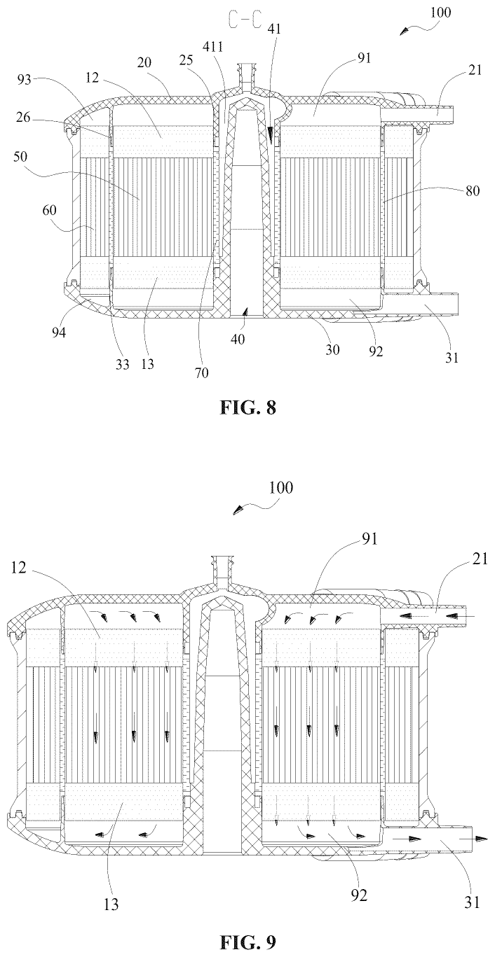

As shown in FIG. 8, a first sealing layer 12 close to the first end cover 20 and a second sealing layer 13 close to the second end cover 30 are provided in the housing 10. The first sealing layer 12 may be formed in the housing 10 close to the first end cover 20, or may be integrally formed in the housing 10 or the first end cover 20.

The first sealing layer 12 and the second sealing layer 13 are formed in the following way. After the oxygenation fiber membranes and the temperature control fiber membranes are wound, the wound oxygenation fiber membranes and temperature control fiber membranes, together with tooling, are placed on a centrifuge. The tooling is connected with a potting device, and the centrifuge is started. Under the action of the centrifugal force, a potting material enters the tooling to pot one end of the oxygenation fiber membranes and the temperature control fiber membranes. After completion, the direction is reversed, the above process is repeated, and the other end of the oxygenation fiber membranes and the temperature control fiber membranes is potted. After cured, the potting material is cut at positions close to outer sides, and at the same time, both ends of the membranes are cut, so that a flat outer surface of the potting material is formed, and end portions of the membranes are exposed. In this way, the sealing layers and the membranes are fabricated.

A first chamber 91 is formed between the first sealing layer 12 and the first end cover 20, and a second chamber 92 is formed between the second sealing layer 13 and the second end cover 30. A first circumferential flange 25 and a second circumferential flange 26 are formed on the first end cover 20, and are respectively located on the inside and outside. A third circumferential flange 33 is formed on the second end cover 30 in correspondence with the second circumferential flange 26. Two ends of the first isolator 70 are respectively connected to the first circumferential flange 25 and the diversion cone 40. Two ends of the second isolator 80 are respectively connected to the second circumferential flange 26 and the third circumferential flange 33.

A cavity between the first sealing layer 12 and the first end cover 20 is divided into two mutually isolated chambers by the second circumferential flange 26, and a cavity between the second sealing layer 13 and the second end cover 30 is divided into two mutually isolated chambers by the third circumferential flange 33. The first chamber 91 is in communication with the first connecting port 21, and the second chamber 92 is in communication with the second connecting port 31. A third chamber 93 is in communication with the third connecting port 22, and a fourth chamber 94 is in communication with the fourth connecting port 32. One end of the oxygenation fiber membranes passes through the first sealing layer 12 to be in communication with the first chamber 91, and the other end of the oxygenation fiber membranes passes through the second sealing layer 13 to be in communication with the second chamber 92. One end of the temperature control fiber membranes passes through the first sealing layer 12 to be in communication with the third chamber, and the other end of the temperature control fiber membranes passes through the second sealing layer 13 to be in communication with the fourth chamber.

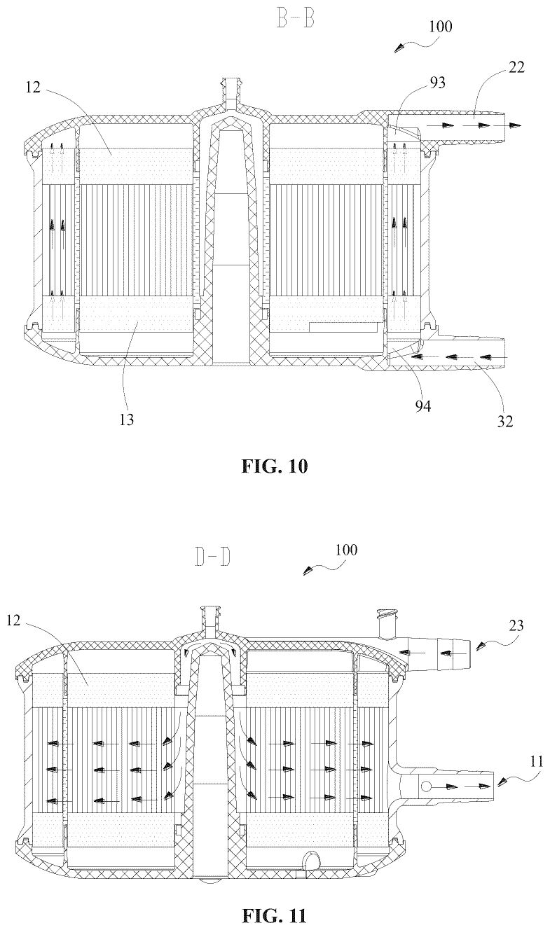

As shown in FIG. 9, solid arrows illustrate flow trajectories of the oxygenation medium. The oxygenation medium enters the first chamber 91 through the first connecting port 21, and enters a port of the oxygenation fiber membranes at the first sealing layer 12. After oxygenation, the oxygenation medium is discharged through another port of the oxygenation fiber membranes at the second sealing layer 13 into the second chamber 92, and is finally discharged through the second connecting port 31. As shown in FIG. 10, solid arrows illustrate flow trajectories of the temperature control medium. The temperature control medium enters the fourth chamber 94 through the fourth connecting port 32, and enters a port of the temperature control fiber membranes at the second sealing layer 13. After temperature control, the temperature control medium is discharged through another port of the temperature control fiber membranes at the first sealing layer 12 into the third chamber 93, and is finally discharged through the third connecting port 22.

The diversion cone 40 is substantially of a tapered conical structure extending in a direction from the second end cover 30 to the first end cover 20. A gap distance between an outer wall of the diversion cone 40 and an inner wall of the first isolator 70 gradually decreases in the direction from the first end cover 20 to the second end cover 30. That is, the gap between the outer wall of the diversion cone 40 and the inner wall of the first isolator 70 gradually shrinks in the flow direction of the blood. As shown in FIG. 11, the gap shrinking downwards can compensate for the pressure of the blood entering the oxygenation module. As described hereinabove, when the blood is flowing, the flow consists of components or suffers resistances in two directions, resulting in pressure loss of forward flow (downward flow as shown in FIG. 11) of the blood. To compensate for this loss, the above-mentioned gap is designed to shrink. According to Bernoulli's principle, the downstream blood with pressure loss regains high pressure with the help of the shrinking gap to enter the oxygenation module. As such, when the blood flows into the oxygenation module, it is possible to achieve uniform pressure as much as possible over a lateral surface of the oxygenation module. In this way, the pressure uniformity of the blood entering the oxygenation module can be maximized, thereby enhancing the oxygenation effect.

The first circumferential flange 25 is connected to the diversion cone 40 through the first isolator 70. Hence, a blood guiding chamber 41, which accommodates the diversion cone 40, is defined by the first circumferential flange 25 and the first isolator 70. The blood guiding chamber 41 includes the blood inlet region 411, as shown in FIG. 7. The blood inlet region 411 is connected to the extension portion 231 of the blood inlet 23, and the extension portion 231 of the blood inlet 23 is eccentric relative to the blood inlet region 411. As shown in FIG. 8, the blood inlet region 411 is a region in the blood guiding chamber 41 between the first end cover 20 and a surface cross section of the first sealing layer 12 away from the first end cover 20. An end of the diversion cone 40 extends into the blood inlet region 411. Similarly, in order to compensate for the pressure drop during the flowing of the blood, a minimum effective flow area of the blood inlet region 411 is larger than or equal to a cross-sectional area of the blood inlet 23. The minimum effective flow area of the blood inlet region 411 refers to an area at the surface cross section (as illustrated by a dotted line in FIG. 8) of the first sealing layer 12 away from the first end cover 20.

As shown in FIG. 11, solid arrows illustrate flow trajectories of the blood. The blood enters the blood inlet region 411 through the blood inlet 23, and the bubbles are separated from the blood under the action of the centrifugal force. The blood flows through the shrinking blood guiding chamber 41 to be compensated for pressure. The blood passes through the first hole of the first isolator 70, and is oxygenated when flowing through the spacing between the oxygenation fiber membranes. Then the blood passes through the second hole of the second isolator 80, and the temperature of the blood is controlled when flowing through the spacing between the temperature control fiber membranes. After that, the blood flows into the gap space between the temperature control module 60 and the housing 10, and finally flows out of the blood outlet 11.

When the volume V and the height H of the oxygenation fiber membranes remain constant, the inner diameter r of the oxygenation layer 50 can be reduced by arranging the oxygenation module 50 on the inside, leading to a longer flow length L of the blood, thereby enhancing the oxygenation efficiency. Further, the blood pressure drop is kept within a desirable range by adjusting the ratio L/H. It is found in researches and experiments that, when the ratio L/H of the thickness L and the height H of the oxygenation module 50 is between 0.525 and 1.562, the oxygenation efficiency and the pressure drop of the oxygenator can be balanced to the greatest extent, that is, the oxygenation efficiency is maximized while reducing the blood pressure drop as much as possible.

It should be noted that, any numerical value in the present disclosure includes all values increasing by one unit from the lower limit to the upper limit, as long as there is an interval of at least two units between any lower value and any higher value.

For example, the ratio L/H is between 0.525 and 1.562, further between 0.575 and 1.512, even further between 0.625 and 1.462, and still further between 0.700 and 1.200, the purpose of which is to describe values not explicitly listed hereinabove, such as 0.701, 0.786, 0.851, 0.889, 0.925, 0.963, 1.035, 1.152, 1.176 and the like.

As described hereinabove, an example range with an interval unit of 0.05 does not preclude increases at appropriate intervals, such as 0.01, 0.02, 0.03, 0.04, 0.06, 0.1, 0.2, 0.3, 0.4, 0.5, and other values. These are merely intended to describe clearly, and it may be understood that, all possible combinations of values listed between the lower limit and the upper limit are explicitly described in a similar manner in this specification.

Other limitations on numerical ranges herein may refer to the above description, and will not be repeated.

According to the description of the blood flow passage hereinabove, after passing through the blood guiding chamber 41, the blood flows through the holes of the first isolator 70 and the second isolator 80 in the listed sequence. In some embodiments, a ratio of a volume of the first hole to a volume of a space occupied by the first isolator 70 is α1, and a ratio of a volume of the second hole to a volume of a space occupied by the second isolator 80 is α2 (which are referred to as porosities hereinafter). A diameter of the hole on each isolator should not be excessively small; otherwise, the hole applies a greater resistance to the blood flowing through, causing higher pressure drop. Certainly, the diameter of the hole on each isolator should not be excessively large; otherwise, the perfusion volume of the blood may be increased.

Therefore, to balance the pressure drop and the perfusion volume, in the present embodiment, a value of α1 is between 0.452 and 0.951, and a value of α2 is between 0.311 and 0.849. Further, the value of α1 is between 0.552 and 0.941, and the value of α2 is between 0.411 and 0.839. Even further, the value of α1 is between 0.652 and 0.931, and the value of α2 is between 0.511 and 0.829. Still further, the value of α1 is between 0.752 and 0.921, and the value of α2 is between 0.611 and 0.819.

For the porosities of the two isolators 70, 80, both the pressure drop and the perfusion volume are expected to be taken into account. Since the two isolators are respectively located at an inner layer and an outer layer (for embodiments with cylindrical oxygenation module and temperature control module), the two isolators 70, 80 have different porosities. The first isolator 70 on the inside has smaller volume and circumferential area than those of the second isolator 80 on the outside. Therefore, the porosity α1 of the first isolator 70 is higher than the porosity α2 of the second isolator 80, so that the blood flows through the two isolators 70, 80 at same speeds.

It should be noted that, with the comparative relationships and numeral ranges of the porosities of the two isolators 70, 80, not only can the blood pressure drop and the blood perfusion volume of the oxygenator be reduced during operation, but also the volume of a priming fluid at the priming stage before the operation of the oxygenator can be reduced. In this way, the priming time can be decreased, thereby deploying the device quickly.

As shown in FIGS. 8 to 10, in order to further reduce the blood perfusion volume, a ratio of a volume of a part of the diversion cone 40 extending into the blood inlet region 411 to a volume of the blood inlet region 411 is between 0.293 and 0.726, further between 0.393 and 0.626, even further between 0.433 and 0.596, and still further between 0.493 and 0.586. As such, most of a space of the blood inlet region 411 is occupied by the diversion cone 40, so that the blood perfusion volume is decreased.

The above arrangement of the diversion cone 40 can also reduce the volume of the priming fluid at the priming stage, which is not described in detail herein.

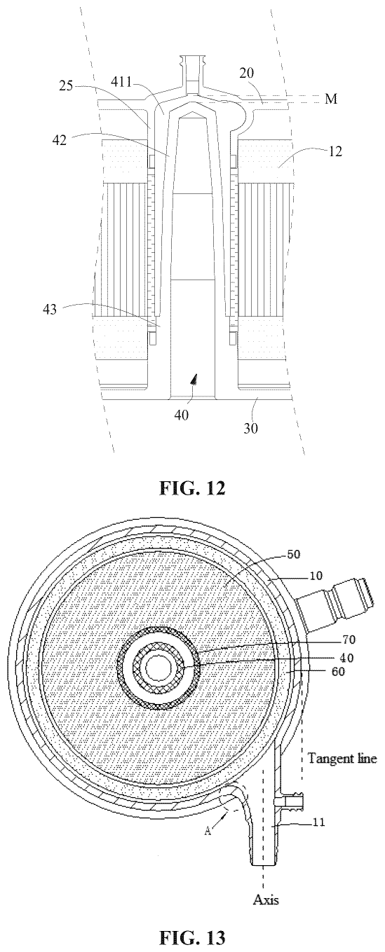

As shown in FIG. 12, the diversion cone 40 includes two cone sections, namely a first cone section 42 that is close to the first end cover 20 and a second cone section 43 that is close to the second end cover 30 and is connected to the first cone section 42. A part of the first cone section 42 is located in the blood inlet region 411, and a cone tip of the first cone section 42 passes through the first sealing layer 12 into the first circumferential flange 25. The second cone section 43 is integrally formed on the second end cover 30. One part of the second cone section 43 is located in the first isolator 70, and the other part of the second cone section 43 (a lower part shown in FIG. 12) is located outside the first isolator 70.

A distance M is present between the cone tip of the first cone section 42 and a top of the blood inlet region 411 which are spaced apart. A value of M is between 0.012 cm and 0.546 cm, further between 0.062 cm and 0.496 cm, even further between 0.112 cm and 0.446 cm, and still further between 0.212 cm and 0.346 cm. A ratio of the value of M to a height of the first cone section 42 is between 0.009 and 0.237, further between 0.019 and 0.227, even further between 0.069 and 0.177, and still further between 0.1 and 0.2.

The definitions of the distance M between the cone tip of the first cone section 42 and the top of the blood inlet region 411 and the ratio of the distance M to the height of the first cone section 42 are also for reducing the blood perfusion volume and the priming fluid volume, which are not described in detail herein.

Most of the first cone section 42 is located in the blood inlet region 411, and a small part of the first cone section 42 is located in the first isolator 70. The first cone section 42 is for guiding the blood flowing into the blood inlet region 411 (guiding the blood to flow downwards as shown in FIG. 12) and unifying the blood. Most of the second cone section 43 is located in the first isolator 70, and is mainly for forming the shrinking gap described hereinabove together with the first isolator 70, so as to compensate for the pressure loss of the blood entering the oxygenation module.

In view of this, a cone angle θ1 of the first cone section 42 is larger than a cone angle θ2 of the second cone section 43. A gap between the first cone section 42 having the smaller cone angle and the first circumferential flange 25 is larger than a gap between the second cone section 43 having the larger cone angle and the first isolator 70. As described hereinabove, the blood perfusion volume is significantly decreased when the first cone section 42 occupies most of the space of the blood inlet region 411, and is decreased with the distance M between the cone tip of the first cone section 42 and the top of the blood inlet region 411 defined hereinabove. Based on this, the relatively large gap between the first cone section 42 and the first circumferential flange 25 helps to reduce the flow resistance applied to the blood, thereby decreasing the blood pressure drop.

It should be noted that, in the description of configuration of the above embodiment, the oxygenation module 50 and the temperature control module 60 are substantially cylindrical, and the oxygenation module 50 is located on an inner side of the temperature control module 60. In this embodiment, the blood inlet 23 is arranged on the first end cover 20, and is in communication with an inner side wall of the oxygenation module 50 through the blood inlet region 411 and the shrinking gap formed between the diversion cone 40 and the first isolator 70. The blood outlet 11 is arranged on the side wall of the housing 10, and is in communication with an outer side wall of the temperature control module 60 through the gap between the temperature control module 60 and the housing 10. An axial direction of the blood inlet 23 is substantially parallel to axial directions of the oxygenation fiber membranes and the temperature control fiber membranes. Specifically, the axial direction of the blood inlet 23 is substantially perpendicular to an axial direction of the housing 10. The oxygenation fiber membranes and the temperature control fiber membranes are basically arranged vertically in the housing 10, that is, the axial directions of the oxygenation fiber membranes and the temperature control fiber membranes are basically parallel to the axial direction of the housing 10. As shown in FIG. 9, a flow path of the blood on a single side of a cross section is roughly of a “⊏” shape.

Certainly, based on the technical essence of the present disclosure that the oxygenation is performed before the temperature control, other configurations of the oxygenation module 50, the temperature control module 60, and the communication manners among these two working modules, the blood inlet 23, and the blood outlet 11 may be provided in other embodiments, which are not limited to the above embodiment.

For example, in an embodiment, the oxygenation module 50 and the temperature control module 60 are also cylindrical, and the difference lies in that the oxygenation module 50 is on the outside, while the temperature control module 60 is on the inside. Correspondingly, the blood inlet 23 is arranged on the side wall of the housing 10, and is in communication with an outer side wall of the oxygenation module 50 through a gap between the oxygenation module 50 and the housing 10. The blood inlet 11 is arranged on at least one of the two end covers, and is in communication with an inner side wall of the temperature control module 60 through the blood inlet region 411 and the shrinking gap formed between the diversion cone 40 and the first isolator 70. The orientations of the blood inlet 23, the oxygenation fiber membranes and the temperature control fiber membranes are the same as those in the above embodiment, which are not described in detail herein. The flow path of the blood is roughly of a “Γ” shape or a “├” shape.

Alternatively, in another embodiment, the oxygenation module and the temperature control module are of a platy shape, a blocky shape or a layered shape with a certain thickness, and the two modules are stacked. This embodiment differs from the above two embodiments in that, the diversion cone may not be provided. To ensure the degassing, the blood inlet is also eccentrically arranged, so that the blood can form a vortex at the inlet, and the gas can be separated from the blood under the action of the centrifugal force. The blood inlet and the blood outlet are respectively located on both sides or opposite sides of the oxygenation module and the temperature control module. Specifically, the blood inlet and the blood outlet may be respectively arranged on the two end covers, or may be arranged on an outer wall of the housing 10. The blood inlet is in communication with a side wall of the oxygenation module through a gap or space (similar to the blood inlet region 411 described hereinabove) between the oxygenation module and one of the end covers such as the first end cover, and the blood outlet is in communication with a side wall of the temperature control module through a gap or space between the temperature control module and the other one of the end covers such as the second end cover. The flow path of the blood is roughly of a “|” shape or a “-” shape.

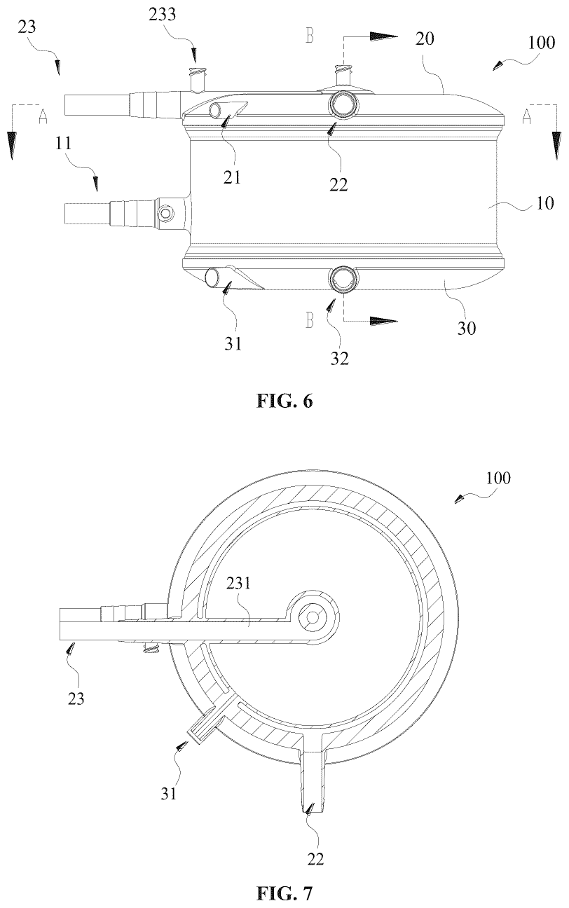

In the embodiment shown in FIGS. 4 to 12, the blood outlet 11 is arranged on the side wall of the housing 10, and an axis of the blood outlet 11 crosses a central axis of the housing 10. Another arrangement of the blood outlet 11 is shown in FIG. 13. In this embodiment, the axis of the blood outlet 11 is located on an inner side of a tangent line. The tangent line and the axis of the blood outlet 11 are located on the same side of the central axis of the housing 11. The tangent line is parallel to the axis of the blood outlet 11, and is tangent to the outer wall of the housing 10. A distance between the axis and the tangent line is determined according to practice. For example, the distance between the axis and the tangent line is between 2 cm and 10 cm, further between 3 cm and 9 cm, even further between 4 cm and 8 cm, and still further between 5 cm and 7 cm. The arrangement of the blood outlet 11 may be understood as that, the blood outlet 11 is offset inwards from a position, which is on the housing 10 and tangent to the housing 10, by a certain distance.

It should be noted that, a tangential blood outlet 11 has a better hydraulic performance compared with the blood outlet 11 shown in FIGS. 4 to 12, which has been described in existing embodiments including but not limited to the disclosure US20200237994A1, and is not described in detail herein.

It should be noted that, it can be known from basic geometric knowledge that, a sharp corner may be formed between the tangential blood outlet 11 and the housing 10. The blood flowing at a relatively low speed may stagnate at this corner, causing thrombosis. In practice, the amount of this part of low-speed blood is relatively small. However, once a thrombus forms, it will further impede the low-speed blood out, thereby aggravating the formation and enlargement of the thrombus. Additionally, once the thrombus is washed away by high-speed blood and enters the circulation between the patient and an extracorporeal device such as a blood pump (i.e. the extracorporeal circulation), it may cause harm to the patient, such as causing organ ischemia, limb necrosis or the like if the thrombus lodges in the blood vessels of the patient.

If the blood outlet 11 is strictly tangential, it is impossible to design a buffer structure, such as a round corner or an arc structure, at the sharp corner between the blood outlet 11 and the housing 10. The reason is, the housing 10 and the blood outlet 11 on the housing 10 are formed by molding, and are expected to be demolded after fabrication. There is no space for demolding on a side opposite to the sharp corner if the blood outlet 11 is strictly tangential, and thus the above buffer structure is infeasible.

On the contrary, in this embodiment, the tangential blood outlet 11 is offset inwards in a parallel manner. As described hereinabove, the amount of the low-speed blood is relatively small. Hence, the offset arrangement will not cause a loss of the hydraulic performance (depending on the offset distance which may not be too large). With the above offset arrangement, a space is reserved for demolding, so that it is possible to provide a round corner or an arc transition at an acute corner section A formed between the blood outlet 11 and the housing 10.

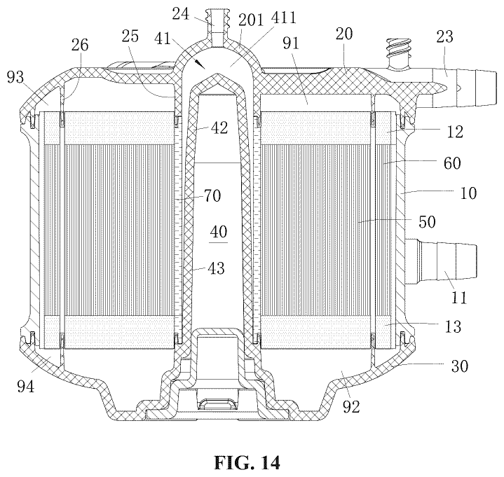

In the embodiment shown in FIGS. 4 to 12, the structure, which is formed on the first end cover 20 in communication with the blood inlet 23 and is provided with the gas vent 24, is basically of a low-profile conical shape. In an embodiment shown in FIG. 14, differing from the above embodiment, this structure 201 protrudes outwards and is substantially of a dome shape or a semi-spherical shape. Compared with the low-profile conical structure, the protruding dome-shaped structure 201 has a smoother inner wall, and a distance to the cone tip of the diversion cone 40 is extended to some extent. It is found in practice that, this structure 201 will not significantly increase the blood perfusion volume. Furthermore, the distance between this structure 201 and the cone tip of the diversion cone 40 is increased, so as to provide time for the released bubbles to rise, making the degassing more thorough.

Further, in the embodiment shown in FIGS. 4 to 12, the second isolator 80 is provided between the oxygenation module 50 and the temperature control module 60. The second isolator 80 is mainly for the fabrication process of the temperature control module 60, which is described hereinabove and will not be described in detail. In the embodiment shown in FIG. 14, differing from the above embodiment, no any structure is provided between the oxygenation module 50 and the temperature control module 60. That is, the second isolator 80 in the above embodiment may be removed. In a case that the second isolator 80 is removed, the fabrication process of the temperature control module 60 is roughly as follows. After the temperature control fiber membranes are wound with a fixture to finish the fabrication of the temperature control module, the fixture is pulled out, and then the wound cylindrical temperature control module 60 is arranged around the oxygenation module 50.

Since there are no physical structural obstructions, such as the second isolator 80, between the oxygenation module 50 and the temperature control module 60, a clearance between the oxygenation module 50 and the temperature control module 60 can be very small. In practice, without the limitation by the second isolator 80 or other physical structures, the fiber membranes of the two modules may come loose and expand to contact each other, filling the space originally occupied by the second isolator 80. Therefore, this structural design not only reduces the blood perfusion volume, but also more greatly lowers the blood pressure drop.

The above embodiments only show several implementations of the present invention. The description thereof is relatively specific and detailed, which should not be construed as limitations to the scope of the present invention. It should be noted that, for those skilled in the art, a few of modifications and improvements may be made to the present invention without departing from the concept of the present invention, and these modifications and improvements are also deemed to fall into the protection scope of the present invention. Therefore, the protection scope of the present invention is defined by the claims.

1. An oxygenator, comprising:
a housing;
a first end cover, which is arranged at a first end of the housing and is provided with a first connecting port;
a second end cover, which is arranged at a second end of the housing, and is provided with a second connecting port, wherein one of the first connecting port and the second connecting port is an oxygenation medium inlet, and the other one of the first connecting port and the second connecting port is an oxygenation medium outlet;
a first sealing layer, which is close to the first end cover, wherein at least a part of the first sealing layer is formed in the housing, a first chamber is defined by the first sealing layer and the first end cover, and the first chamber is in communication with the first connecting port;
a second sealing layer, which is close to the second end cover, wherein at least a part of the second sealing layer is formed in the housing, a second chamber is defined by the second sealing layer and the second end cover, and the second chamber is in communication with the second connecting port;
an oxygenation module, which is arranged in the housing, wherein a side wall of the oxygenation module is in communication with a blood inlet, the oxygenation module comprises an oxygenation fiber membrane, and two ends of the oxygenation fiber membrane pass through the first sealing layer and the second sealing layer to be in communication with the first chamber and the second chamber respectively; and
a temperature control module, which is arranged in the housing and is located downstream of the oxygenation module in a flow direction of blood, wherein a side wall of the temperature control module is in communication with a blood outlet.

2. The oxygenator according to claim 1, wherein
the first end cover is further provided with a third connecting port, and the second end cover is further provided with a fourth connecting port;
one of the third connecting port and the fourth connecting port is a temperature control medium inlet, and the other one of the third connecting port and the fourth connecting port is a temperature control medium outlet;
a third chamber is further defined by the first sealing layer and the first end cover, and is fluidly isolated from the first chamber, and a fourth chamber is further defined by the second sealing layer and the second end cover, and is fluidly isolated from the second chamber; and
the temperature control module comprises a temperature control fiber membrane, and two ends of the temperature control fiber membrane pass through the first sealing layer and the second sealing layer to be in communication with the third chamber and the fourth chamber respectively.

3. The oxygenator according to claim 2, wherein
the blood inlet is provided on the first end cover, the oxygenation module is substantially of a cylindrical structure, and an inner side wall of the oxygenation module is in communication with the blood inlet;
the temperature control module is substantially of a cylindrical structure, and is arranged on an outer side of the oxygenation module; and
an outer side wall of the temperature control module is spaced apart from an inner side wall of the housing to form a gap space therebetween, and the gap space is in communication with the blood outlet.

4. The oxygenator according to claim 1, wherein
the blood outlet is provided on a side wall of the housing, and an axis of the blood outlet is located on an inner side of a tangent line; and
the tangent line and the axis are located on the same side of a central axis of the housing, and the tangent line is parallel to the axis and is tangent to an outer wall of the housing.

5. The oxygenator according to claim 4, wherein an acute corner section is formed between the blood outlet and the housing, and a round corner or an arc transition is provided at the acute corner section.

6. The oxygenator according to claim 1, wherein a substantially dome-shaped structure that protrudes outwards is formed on the first end cover, the blood inlet is in communication with the dome-shaped structure, and the dome-shaped structure is provided with a gas vent.

7. The oxygenator according to claim 1, wherein no any structure is provided between the oxygenation module and the temperature control module.

8. The oxygenator according to claim 1, wherein a ratio L/H of a thickness L of the oxygenation module in a radial direction to a height H of the oxygenation module in an axial direction is between 0.525 and 1.562.

9. The oxygenator according to claim 2, wherein
a first isolator is provided in the housing, and the oxygenation fiber membrane is wound around the first isolator;
the first isolator is of a hollow cylindrical structure, an internal space of the first isolator is in communication with the blood inlet, and a side wall of the first isolator is provided with a first hole for the blood to pass through;
a second isolator is provided in the housing and is located between the oxygenation module and the temperature control module, the temperature control fiber membrane is wound around the second isolator, and a side wall of the second isolator is provided with a second hole for the blood to pass through; and
a ratio of a volume of the first hole to a volume of a space occupied by the first isolator is α1, a ratio of a volume of the second hole to a volume of a space occupied by the second isolator is α2, and α1>α2.

10. The oxygenator according to claim 9, wherein a diversion cone is provided in the housing and passes through the first isolator, and a gap distance between an outer wall of the diversion cone and an inner wall of the first isolator gradually decreases in a direction from the first end cover to the second end cover.

11. The oxygenator according to claim 10, wherein
a circumferential flange is formed on the first end cover and extends to the first sealing layer, one end of the first isolator is connected to the diversion cone, and the other end of the first isolator is connected to the circumferential flange;
a blood guiding chamber is defined by the circumferential flange and the first isolator, and accommodates the diversion cone; and
the blood guiding chamber comprises a blood inlet region, and a part of the diversion cone extends into the blood inlet region, wherein the blood inlet region is a region in the blood guiding chamber between the first end cover and a surface cross section of the first sealing layer away from the first end cover.

12. The oxygenator according to claim 11, wherein a ratio of a volume of the part of the diversion cone extending into the blood inlet region to a volume of the blood inlet region is between 0.293 and 0.726.

13. The oxygenator according to claim 12, wherein the diversion cone comprises a first cone section that is close to the first end cover, a cone tip of the first cone section passes through the first sealing layer into the circumferential flange, and a distance between the cone tip and a top of the blood inlet region is between 0.012 cm and 0.546 cm.

14. The oxygenator according to claim 13, wherein a ratio of the distance between the cone tip of the first cone section and the top of the blood inlet region to a height of the first cone section is between 0.009 and 0.237.

15. The oxygenator according to claim 13, wherein the diversion cone further comprises a second cone section that is close to the second end cover and is connected to the first cone section, a part of the second cone section is located in the first isolator, and a cone angle of the first cone section is greater than a cone angle of the second cone section.

16. The oxygenator according to claim 11, wherein a minimum effective flow area of the blood inlet region is larger than or equal to a cross-sectional area of the blood inlet.

17. The oxygenator according to claim 3, wherein a ratio L/H of a thickness L of the oxygenation module in a radial direction to a height H of the oxygenation module in an axial direction is between 0.525 and 1.562.

18. The oxygenator according to claim 5, wherein a ratio L/H of a thickness L of the oxygenation module in a radial direction to a height H of the oxygenation module in an axial direction is between 0.525 and 1.562.

19. The oxygenator according to claim 1, wherein no structure is provided between the oxygenation module and the temperature control module.

20. A blood oxygenator, comprising:
a housing comprising a first end and a second end;
a first end cover at the first end of the housing, wherein the first end cover comprises a first connecting port;
a second end cover at the second end of the housing, wherein the second end cover comprises a second connecting port, wherein one of the first connecting port and the second connecting port is an oxygenation medium inlet, and the other one of the first connecting port and the second connecting port is an oxygenation medium outlet;
a first sealing layer adjacent the first end cover, wherein at least a part of the first sealing layer is formed in the housing, wherein a first chamber is defined by the first sealing layer and the first end cover, and wherein the first chamber is in communication with the first connecting port;
a second sealing layer adjacent the second end cover, wherein at least a part of the second sealing layer is formed in the housing, wherein a second chamber is defined by the second sealing layer and the second end cover, and wherein the second chamber is in communication with the second connecting port;
an oxygenation module arranged in the housing, wherein the oxygenation module comprises:
a side wall in communication with a blood inlet; and
an oxygenation fiber membrane, wherein a first end of the oxygenation fiber membrane passes through the first sealing layer to be in communication with the first chamber and a second end of the oxygenation fiber membrane passes through the second sealing layer to be in communication with the second chamber; and
a temperature control module arranged in the housing, wherein the temperature control module is located downstream of the oxygenation module in a flow direction of blood, and wherein a side wall of the temperature control module is in communication with a blood outlet.
